SEQUENCE LISTING (66 sequences)

SEQ ID NO: 1 - DNA, 657 bp, Fungia sp.
  CDS (1)..(654)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccaagggcgggccaatgcctttcgcgtttgacttagtgtcacacgtgttctgttacggccacagaccttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggaggcggcaatcacaaatgccaattcaagactacttacaaggcggcaaaaaagattcttaaaatgccaggaagccattacatcagccatcgcctcgtcaggaaaaccgaaggcaacattactgagctggtagaagatgcagtagctcattcctga

SEQ ID NO: 2 - protein, 218 aa, Fungia sp.
MVSVIKPEMKMRYYMDGSVNGHEFTIEGEGTGRPYEGHQEMTLRVTMAKGGPMPFAFDLVSHVFCYGHRPFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKKILKMPGSHYISHRLVRKTEGNITELVEDAVAHS

SEQ ID NO: 3 - DNA, 657 bp, Fungia sp.
  CDS (1)..(654)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagagtatttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggaggcggcaatcacaaatgccaattcaagactacttacaaggcggcaaaagagattcttgaaatgccaggagaccattacatcggccatcgcctcgtcaggaaaaccgaaggcaacattactgagctggtagaagatgcagtagctcattcctaa

SEQ ID NO: 4 - protein, 218 aa, Fungia sp.
MVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITELVEDAVAHS

SEQ ID NO: 5 - DNA, 657 bp, Fungia sp.
  CDS (1)..(654)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagagcttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggaggcggcaatcacaaatgccaattcaagactacttacaaggcggcaaaagagattcttgaaatgccaggagaccattacatcgggcatcgcctcgtcaggaaaaccgaaggcaacattactgagctggtagaagatgcagtagctcattcctaa

SEQ ID NO: 6 - protein, 218 aa, Fungia sp.
MVSVIKPEMKMRYYMDGSVNGHEFTIEGEGTGRPYEGHQEMTLRVTMAEGGPMPFAFDLVSHVFCYGHRAFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITELVEDAVAHS

SEQ ID NO: 7 - DNA, 657 bp, Fungia sp.
  CDS (1)..(654)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagatcttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggaggcggcaatcacaaatgccaattcaagactacttacaaggcggcaaaagagattcttgaaatgccaggagaccattacatcggccatcgcctcgtcaggaaaaccgaaggcaacattactgagctggtagaagatgcagtagctcattcctaa

SEQ ID NO: 8 - protein, 218 aa, Fungia sp.
MVSVIKPEMKMRYYMDGSVNGHEFTIEGEGTGRPYEGHQEMTLRVTMAEGGPMPFAFDLVSHVFCYGHRSFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITELVEDAVAHS

SEQ ID NO: 9 - DNA, 657 bp, Fungia sp.
  CDS (1)..(654)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagatgttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggaggcggcaatcacaaatgccaattcaagactacttacaaggcggcaaaagagattcttgaaatgccaggagaccattacatcggccatcgcctcgtcaggaaaaccgaaggcaacattactgagctggtagaagatgcagtagctcattcctaa

SEQ ID NO: 10 - protein, 218 aa, Fungia sp.
MVSVIKPEMKMRYYMDGSVNGHEFTIEGEGTGRPYEGHQEMTLRVTMAEGGPMPFAFDLVSHVFCYGHRCFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITELVEDAVAHS

SEQ ID NO: 11 - DNA, 657 bp, Fungia sp.
  CDS (1)..(654)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagaacttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggaggcggcaatcacaaatgccaattcaagactacttacaaggcggcaaaagagattcttgaaatgccaggagaccattacatcggccatcgcctcgtcaggaaaaccgaaggcaacattactgagctggtagaagatgcagtagctcattcctaa

SEQ ID NO: 12 - protein, 218 aa, Fungia sp.
MVSVIKPEMKMRYYMDGSVNGHEFTIEGEGTGRPYEGHQEMTLRVTMAEGGPMPFAFDLVSHVFCYGHRTFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGGNHKCQFKTTYKAAKEILEMPGDHYIGHRLVRKTEGNITELVEDAVAHS

SEQ ID NO: 13 - DNA, 678 bp, Fungia sp.
  CDS (1)..(678)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagagtatttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggagggaattctgcagatggtggaggcggttcaggcggaggtggctctggcggtggcggatcgatccatcacactggcggccgcgcccagctcgaaaaggagctgcaagccctggagaaggagaacgcccagctcgaatgggagctccaggccctggagaaggagctggcccagaag

SEQ ID NO: 14 - protein, 226 aa, Fungia sp.
MVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGNSADGGGGSGGGGSGGGGSIHHTGGRAQLEKELQALEKENAQLEWELQALEKELAQK

SEQ ID NO: 15 - DNA, 327 bp, Fungia sp.
  CDS (1)..(327)
atgggcggcaatcacaaatgccaattcaagactacttacaaggcggcaaaagagattcttgaaatgccaggagaccattacatcggccatcgcctcgtcaggaaaaccgaaggcaacattactgagctggtagaagatgcagtagctcattccgggaattctgcagatggtggaggcggttcaggcggaggtggctctggcggtggcggatcgatccatcacactggcggccgcgcccagctcaagaagaagctgcaagccctgaagaagaagaacgcccagctcaagtggaagctccaggccctgaagaagaagctggcccagaag

SEQ ID NO: 16 - protein, 109 aa, Fungia sp.
MGGNHKCQFKTTYKAAKEILEMPGDHYIGHRLVRKTEGNITELVEDAVAHSGNSADGGGGSGGGGSGGGGSIHHTGGRAQLKKKLQALKKKNAQLKWKLQALKKKLAQK

SEQ ID NO: 17 - DNA, 504 bp, Fungia sp.
  CDS (1)..(504)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagagtatttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaagga

SEQ ID NO: 18 - protein, 168 aa, Fungia sp.
MVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEG

SEQ ID NO: 19 - DNA, 153 bp, Fungia sp.
  CDS (1)..(153)
atgggcggcaatcacaaatgccaattcaagactacttacaaggcggcaaaagagattcttgaaatgccaggagaccattacatcggccatcgcctcgtcaggaaaaccgaaggcaacattactgagctggtagaagatgcagtagctcattcc

SEQ ID NO: 20 - protein, 51 aa, Fungia sp.
MGGNHKCQFKTTYKAAKEILEMPGDHYIGHRLVRKTEGNITELVEDAVAHS

SEQ ID NO: 21 - DNA, 678 bp, Fungia sp.
  CDS (1)..(678)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagatgttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggagggaattctgcagatggtggaggcggttcaggcggaggtggctctggcggtggcggatcgatccatcacactggcggccgcgcccagctcgaaaaggagctgcaagccctggagaaggagaacgcccagctcgaatgggagctccaggccctggagaaggagctggcccagaag

SEQ ID NO: 22 - protein, 226 aa, Fungia sp.
MVSVIKPEMKMRYYMDGSVNGHEFTIEGEGTGRPYEGHQEMTLRVTMAEGGPMPFAFDLVSHVFCYGHRCFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGNSADGGGGSGGGGSGGGGSIHHTGGRAQLEKELQALEKENAQLEWELQALEKELAQK

SEQ ID NO: 23 - DNA, 678 bp, Fungia sp.
  CDS (1)..(678)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagatcttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggagggaattctgcagatggtggaggcggttcaggcggaggtggctctggcggtggcggatcgatccatcacactggcggccgcgcccagctcgaaaaggagctgcaagccctggagaaggagaacgcccagctcgaatgggagctccaggccctggagaaggagctggcccagaag

SEQ ID NO: 24 - protein, 226 aa, Fungia sp.
MVSVIKPEMKMRYYMDGSVNGHEFTIEGEGTGRPYEGHQEMTLRVTMAEGGPMPFAFDLVSHVFCYGHRSFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGNSADGGGGSGGGGSGGGGSIHHTGGRAQLEKELQALEKENAQLEWELQALEKELAQK

SEQ ID NO: 25 - DNA, 678 bp, Fungia sp.
  CDS (1)..(678)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagagcttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggagggaattctgcagatggtggaggcggttcaggcggaggtggctctggcggtggcggatcgatccatcacactggcggccgcgcccagctcgaaaaggagctgcaagccctggagaaggagaacgcccagctcgaatgggagctccaggccctggagaaggagctggcccagaag

SEQ ID NO: 26 - protein, 226 aa, Fungia sp.
MVSVIKPEMKMRYYMDGSVNGHEFTIEGEGTGRPYEGHQEMTLRVTMAEGGPMPFAFDLVSHVFCYGHRAFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGNSADGGGGSGGGGSGGGGSIHHTGGRAQLEKELQALEKENAQLEWELQALEKELAQK

SEQ ID NO: 27 - DNA, 678 bp, Fungia sp.
  CDS (1)..(678)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagaacttttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggagggaattctgcagatggtggaggcggttcaggcggaggtggctctggcggtggcggatcgatccatcacactggcggccgcgcccagctcgaaaaggagctgcaagccctggagaaggagaacgcccagctcgaatgggagctccaggccctggagaaggagctggcccagaag

SEQ ID NO: 28 - protein, 226 aa, Fungia sp.
MVSVIKPEMKMRYYMDGSVNGHEFTIEGEGTGRPYEGHQEMTLRVTMAEGGPMPFAFDLVSHVFCYGHRTFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGNSADGGGGSGGGGSGGGGSIHHTGGRAQLEKELQALEKENAQLEWELQALEKELAQK

SEQ ID NO: 29 - DNA, 33 bp, Artificial Sequence
  Description of Artificial Sequence Synthetic primer
ccagagatgaagatgaggtactacatggacggc

SEQ ID NO: 30 - DNA, 27 bp, Artificial Sequence
  Description of Artificial Sequence Synthetic primer
catgagttcacaattgaaggtgaaggc

SEQ ID NO: 31 - DNA, 27 bp, Artificial Sequence
  Description of Artificial Sequence Synthetic primer
gaaggcacaggcagaccttacgaggga

SEQ ID NO: 32 - DNA, 27 bp, Artificial Sequence
  Description of Artificial Sequence Synthetic primer
ccaatgcctttcgcgtttgacttagtg

SEQ ID NO: 33 - DNA, 27 bp, Artificial Sequence
  Description of Artificial Sequence Synthetic primer
ttagtgtcacacgtgttctgttacggc

SEQ ID NO: 34 - DNA, 27 bp, Artificial Sequence
  Description of Artificial Sequence Synthetic primer
gaaaggtcgttggagttcgaagatggt

SEQ ID NO: 35 - DNA, 30 bp, Artificial Sequence
  Description of Artificial Sequence Synthetic primer
gaagatggtgggtccgcttcagtcagtgcg

SEQ ID NO: 36 - DNA, 34 bp, Artificial Sequence
  Description of Artificial Sequence Synthetic primer
agccttagaggaaacaccttctaccacaaatcca

SEQ ID NO: 37 - DNA, 32 bp, Artificial Sequence
  Description of Artificial Sequence Synthetic primer
caaatccaaatttactggggttaactttcctg

SEQ ID NO: 38 - DNA, 30 bp, Artificial Sequence
  Description of Artificial Sequence Synthetic primer
gccgatggtcctatcatgcaaaaccaaagt

SEQ ID NO: 39 - DNA, 45 bp, Artificial Sequence
  Description of Artificial Sequence Synthetic primer
gccgatggtcctatcatgcaaaaccaaagtgttgattgggagcca

SEQ ID NO: 40 - DNA, 33 bp, Artificial Sequence
  Description of Artificial Sequence Synthetic primer
gagaaaattactgccagcgacggagttctgaag

SEQ ID NO: 41 - DNA, 42 bp, Artificial Sequence
  Description of Artificial Sequence Synthetic primer
gatgttacgatgtacctaaaacttgaaggaggcggcaatcac

SEQ ID NO: 42 - DNA, 45 bp, Artificial Sequence
  Description of Artificial Sequence Synthetic primer
cttaaaatgccaggaagccattacatcagccatcgcctcgtcagg

SEQ ID NO: 43 - DNA, 34 bp, Artificial Sequence
  Description of Artificial Sequence Synthetic primer
gatgcagtagctcattccctcgagcaccaccacc

SEQ ID NO: 44 - DNA, 39 bp, Artificial Sequence
  Description of Artificial Sequence Synthetic primer
aaaaagcttaccatggtgagtgtgattaaaccagagatg

SEQ ID NO: 45 - DNA, 36 bp, Artificial Sequence
  Description of Artificial Sequence Synthetic primer
tgcagaattccctccttcaagttttaggtacatcgt

SEQ ID NO: 46 - DNA, 39 bp, Artificial Sequence
  Description of Artificial Sequence Synthetic primer
aaaaagcttaccatgggcggcaatcacaaatgccaattc

SEQ ID NO: 47 - DNA, 33 bp, Artificial Sequence
  Description of Artificial Sequence Synthetic primer
tgcagaattcccggaatgagctactgcatcttc

SEQ ID NO: 48 - protein, 32 aa, Artificial Sequence
  Description of Artificial Sequence Synthetic peptide
RAQLEKELQALEKENAQLEWELQALEKELAQK

SEQ ID NO: 49 - protein, 32 aa, Artificial Sequence
  Description of Artificial Sequence Synthetic peptide
RAQLKKKLQALKKKNAQLKWKLQALKKKLAQK

SEQ ID NO: 50 - protein, 26 aa, Artificial Sequence
  Description of Artificial Sequence Synthetic peptide
GNSADGGGGSGGGGSGGGGSIHHTGG

SEQ ID NO: 51 - DNA, 38 bp, Artificial Sequence
  Description of Artificial Sequence Synthetic primer
aaggagatataccaatggtgagtgtgattaaaccagag

SEQ ID NO: 52 - DNA, 21 bp, Artificial Sequence
  Description of Artificial Sequence Synthetic primer
tattcattacttctgggccag

SEQ ID NO: 53 - DNA, 38 bp, Artificial Sequence
  Description of Artificial Sequence Synthetic primer
aaggagatataccaatgggcaatcacaaatgccaattc

SEQ ID NO: 54 - DNA, 85 bp, Artificial Sequence
  Description of Artificial Sequence Synthetic primer
gaaattaatacgactcactatagggagaccacaacggtttccctctagaaataattttgtttaactttaagaaggagatatacca

SEQ ID NO: 55 - DNA, 30 bp, Artificial Sequence
  Description of Artificial Sequence Synthetic primer
tattcattatccttcaagttttaggtacat

SEQ ID NO: 56 - DNA, 35 bp, Artificial Sequence
  Description of Artificial Sequence Synthetic primer
tattcattaggaatgagctactgcatcttctacca

SEQ ID NO: 57 - DNA, 1080 bp, Fungia sp.
  CDS (1)..(1077)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttctgttacggccacagagtatttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggagggaattctgcagatggtggaggcggttcaggcggaggtggctctggcggtggcggatcgatccatcacactggcggccgcatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaa

SEQ ID NO: 58 - protein, 359 aa, Fungia sp.
MVSVIKPEMKMRYYMDGSVNGHEFTIEGEGTGRPYEGHQEMTLRVTMAEGGPMPFAFDLVSHVFCYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGNSADGGGGSGGGGSGGGGSIHHTGGRMSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 59 - DNA, 1020 bp, Fungia sp.
  CDS (1)..(1017)
atgggcggcaatcacaaatgccaattcaagactacttacaaggcggcaaaagagattcttgaaatgccaggagaccattacatcggccatcgcctcgtcaggaaaaccgaaggcaacattactgagctggtagaagatgcagtagctcattccgggaattctgcagatggtggaggcggttcaggcggaggtggctctggcggtggcggatcgatccatcacactggcggccgcatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaacaggagtacagctgtgtagtaaagatgccttctggtgaatttgcacgtatatgccgagatctcagccatattggagatgctgttgtaatttcctgtgcaaaagacggagtgaaattttctgcaagtggagaacttggaaatggaaacattaaattgtcacagacaagtaatgtcgataaagaggaggaagctgttaccatagagatgaatgaaccagttcaactaacttttgcactgaggtacctgaacttctttacaaaagccactccactctcttcaacggtgacactcagtatgtctgcagatgtaccccttgttgtagagtataaaattgcggatatgggacacttaaaatactacttggctcccaagatcgaggatgaagaaggatcttaa

SEQ ID NO: 60 - protein, 339 aa, Fungia sp.
MGGNHKCQFKTTYKAAKEILEMPGDHYIGHRLVRKTEGNITELVEDAVAHSGNSADGGGGSGGGGSGGGGSIHHTGGRMFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 61 - DNA, 1080 bp, Fungia sp.
  CDS (1)..(1077)
atggtgagtgtgattaaaccagagatgaagatgaggtactacatggacggctccgtcaatgggcatgagttcacaattgaaggtgaaggcacaggcagaccttacgagggacatcaagagatgacactacgcgtcacaatggccgagggcgggccaatgcctttcgcgtttgacttagtgtcacacgtgttcgcttacggccacagagtatttactaaatatccagaagagataccagactatttcaaacaagcatttcctgaaggcctgtcatgggaaaggtcgttggagttcgaagatggtgggtccgcttcagtcagtgcgcatataagccttagaggaaacaccttctaccacaaatccaaatttactggggttaactttcctgccgatggtcctatcatgcaaaaccaaagtgttgattgggagccatcaaccgagaaaattactgccagcgacggagttctgaagggtgatgttacgatgtacctaaaacttgaaggagggaattctgcagatggtggaggcggttcaggcggaggtggctctggcggtggcggatcgatccatcacactggcggccgcatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaa

SEQ ID NO: 62 - protein, 359 aa, Fungia sp.
MVSVIKPEMKMRYYMDGSVNGHEFTIEGEGTGRPYEGHQEMTLRVTMAEGGPMPFAFDLVSHVFAYGHRVFTKYPEEIPDYFKQAFPEGLSWERSLEFEDGGSASVSAHISLRGNTFYHKSKFTGVNFPADGPIMQNQSVDWEPSTEKITASDGVLKGDVTMYLKLEGGNSADGGGGSGGGGSGGGGSIHHTGGRMSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 63 - DNA, 35 bp, Artificial Sequence
  Description of Artificial Sequence Synthetic primer
actggcggccgcatgtcagaaccggctggggatgt

SEQ ID NO: 64 - DNA, 33 bp, Artificial Sequence
  Description of Artificial Sequence Synthetic primer
gggctcgagttagggcttcctcttggagaagat

SEQ ID NO: 65 - DNA, 35 bp, Artificial Sequence
  Description of Artificial Sequence Synthetic primer
actggcggccgcatgttcgaggcgcgcctggtcca

SEQ ID NO: 66 - DNA, 36 bp, Artificial Sequence
  Description of Artificial Sequence Synthetic primer
gggctcgagttaagatccttcttcatcctcgatctt

(19) United States
(12) Patent Application Publication
(10) Pub. No.: US 2007/0031912 A1
(43) Pub. Date: Feb. 8, 2007

(54) Method for analysis of protein interaction using fluorescent protein

(75) Inventors: Atsushi Miyawaki, Saitama (JP); Satoshi Karasawa, Tokyo (JP)

Correspondence Address: GREENBLUM & BERNSTEIN, P.L.C., 1950 ROLAND CLARKE PLACE, RESTON, VA, 20191, US

(73) Assignees: RIKEN, Saitama (JP); MEDICAL & BIOLOGICAL LABORATORIES CO., LTD., Aichi (JP)

(21) Appl. No.: 11/390,215

(22) Filed: Mar. 28, 2006

(30) Foreign Application Priority Data
Mar. 29, 2005 (JP) 2005-093541
Mar. 27, 2006 (JP) 2006-084809

(51) Int. Cl. C12Q 1/37

(52) U.S. Cl. 435/23

(57) ABSTRACT

An object of the present invention is to provide a method for analyzing the protein interaction, wherein the information about time can be obtained and the movement of protein can be monitored. The present invention provides a method for analyzing interaction between a first test protein and a second test protein which comprises the steps of: splitting a fluorescent protein capable of emitting different color of fluorescence according to passage of time into an N-terminal fragment and a C-terminal fragment; allowing the first test protein to interact with the second test protein by making coexist a fusion protein of the N-terminal fragment with the first test protein and another fusion protein of the C-terminal fragment with the second test protein; and detecting the change in the fluorescent light due to the interaction.

### TECHNICAL FIELD

The present invention relates a method for analysis of protein interaction using a fluorescent protein and a kit for the method for analysis

### BACKGROUND ART

Green fluorescent protein (GFP) derived from jelly fish, Aequorea Victoria, has been used in many biological systems. Recently various mutant GFPs have been produced with changed color, improved folding characteristic, higher luminance, modified pH sensitivity and the like by the random mutagenesis and the semi-rational mutagenesis method. Other protein is fused with a fluorescent protein such as GFP and the like by the genetic recombination technology to monitor the expression and transport of the protein.

On the other hand, the technique of the protein complementation has been used for a long time. This is a method for splitting a protein and then putting back together again. The protein complementation technique is applied on GFP derived from Aequorea Victoria. Namely, GFP is expressed in divided two parts and then the fluorescence is measured by combining the two parts. Problems of this system include: (1) since no information about time is available, it is not known when the interaction (binding) takes place; (2) relating to (1) described above, in the case of a molecule which moves by binding, the movement cannot be monitored because there is no record left after the binding; and the like. Hu C D, Kerppola T K, Nat Biotechnol. May 2003; 21(5): 539-45, and Hu C D, Chinenov Y, Kerppola T K, Mol Cell. April 2002; (9): 789-98

### DISCLOSURE OF THE INVENTION

An object of the present invention is to provide a method for analyzing the protein interaction, wherein the information about time can be obtained and the movement of protein can be monitored.

To achieve the above object, the present inventors have investigated vigorously and found the followings. An N-terminal fragment and a C-terminal fragment of a fluorescent protein, capable of emitting different fluorescent color according to passage of time, were expressed separately and then fluorescence is measured by combining these two fragments. Thus, it became possible to detect the time course of the interaction of the 2 test proteins by means of the fluorescent ratio. It has been also proven that the movement of these proteins after the binding can be monitored. The present invention has been completed based on these findings.

Specifically, the present invention provides a method for analyzing interaction between a first test protein and a second test protein which comprises the steps of: splitting a fluorescent protein capable of emitting different color of fluorescence according to passage of time into an N-terminal fragment and a C-terminal fragment; allowing the first test protein to interact with the second test protein by making coexist a fusion protein of the N-terminal fragment with the first test protein and another fusion protein of the C-terminal fragment with the second test protein; and detecting the change in the fluorescent light due to the interaction.

The fluorescent protein capable of emitting different color of fluorescence according to passage of time is preferably a fluorescent protein having an amino acid sequence of SEQ ID NO: 2 in which the 70^{th} amino acid, proline, is substituted with another amino acid.

More preferably the fluorescent protein capable of emitting different color of fluorescence according to passage of time is any of the following proteins:
(1) a fluorescent protein of an amino acid sequence represented by any of SEQ ID NO: 4, 6, 8, 10 and 12; or
(2) a fluorescent protein of an amino acid sequence represented by any of SEQ ID NO: 4, 6, 8, 10 and 12 wherein one or several amino acid are deleted, substituted and/or added, which has fluorescence characteristic which is changed from green color to orange color according to passage of time.

According to another aspect of the present invention, a kit for analyzing interaction between proteins is provided which includes a combination of a gene encoding an N-terminal fragment of a fluorescent protein having an amino acid sequence represented by SEQ ID NO: 2 in which the 70^{th} amino acid, proline, is substituted with another amino acid and a gene encoding a C-terminal fragment of said fluorescent protein.

Preferably the aforementioned fluorescent protein is any of the following proteins:
(1) a fluorescent protein of an amino acid sequence represented by any of SEQ ID NO: 4, 6, 8, 10 and 12; or
(2) a fluorescent protein of an amino acid sequence represented by any of SEQ ID NO: 4, 6, 8, 10 and 12 wherein one or several amino acid are deleted, substituted and/or added, which has fluorescence characteristic which is changed from green color to orange color according to passage of time.

### BRIEF DESCRIPTION OF THE DRAWINGS

FIG. 1 shows an outline of the method of the present invention for analyzing interaction between proteins.

FIG. 2 shows a method for obtaining the data of the interaction between mKO-FM14-N-LZA and mKO-FM14-C-LZB.

FIG. 3 shows the results by dual wave length excitation and dual wave length photometry. The solid line represents a mixture of mKO-FM14-N-LZA and mKO-FM14-C-LZB and the dotted line represents a mixture of mKO-FM14-N and mKO-FM14-C.

FIG. 4 shows the results by dual wave length excitation and single wave length photometry. The solid line represents a mixture of mKO-FM14-N-LZA and mKO-FM14-C-LZB and the dotted line represents a mixture of mKO-FM14-N and mKO-FM14-C.

FIG. 5 shows the results by dual wave length excitation and dual wave length photometry of the interaction. mKO-FM14-N-LZA and mKO-FM14-C-LZB were mixed.

FIG. 6 shows the results by dual wave length excitation and dual wave length photometry of the interaction. mKO-FM5-N-LZA and mKO-FM14-C-LZB were mixed.

FIG. 7 shows the results by dual wave length excitation and dual wave length photometry of the interaction. mKO-FM3-N-LZA and mKO-FM14-C-LZB were mixed.

FIG. 8 shows the results by dual wave length excitation and dual wave length photometry of the interaction. mKO-FM20-N-LZA and mKO-FM14-C-LZB were mixed.

FIG. 9 shows the results by dual wave length excitation and dual wave length photometry of the interaction. mKO-FM24-N-LZA and mKO-FM14-C-LZB were mixed.

FIG. 10 shows the results by dual wave length excitation and dual wave length photometry of the interaction. Change of Ratio (560 nm/509 nm) of each variant according to time is shown.

FIG. 11 shows the results by dual wave length excitation and single wave length photometry of the interaction. mKO-FM14-N-LZA and mKO-FM14-C-LZB were mixed.

FIG. 12 shows the results by dual wave length excitation and single wave length photometry of the interaction. mKO-FM5-N-LZA and mKO-FM14-C-LZB were mixed.

FIG. 13 shows the results by dual wave length excitation and single wave length photometry of the interaction. mKO-FM3-N-LZA and mKO-FM14-C-LZB were mixed.

FIG. 14 shows the results by dual wave length excitation and single wave length photometry of the interaction. mKO-FM20-N-LZA and mKO-FM14-C-LZB were mixed.

FIG. 15 shows the results by dual wave length excitation and single wave length photometry of the interaction. mKO-FM24-N-LZA and mKO-FM14-C-LZB were mixed.

FIG. 16 shows the results by dual wave length excitation and single wave length photometry of the interaction. Change of Ratio (548 nm/500 nm) of each variant according to time is shown.

FIG. 17 shows fluorescent images of cells in orange and green after 8 hours of transfecting the genes of mKO-FM14-N-p21 and mKO-FM14-C-PCNA.

FIG. 18 shows fluorescent images of cells in orange and green after 22 hours of transfecting the genes of mKO-FM14-N-p21 and mKO-FM14-C-PCNA.

FIG. 19 shows fluorescent images of cells in green after 24 hours of transfecting the genes of mKG-N-p21-pCDNA3 and mKO-FM14-C-PCNA-pCDNA3.

### BEST MODE FOR CARRYING OUT THE INVENTION

The embodiments of the present invention will be described in detail below.

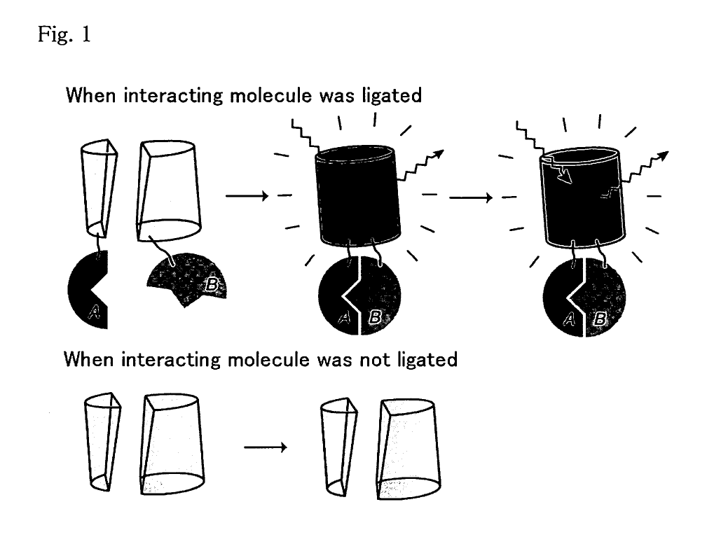

In the present invention, mutations were introduced in fluorescent protein monomer of Kusabira-Orange (mKO) to produce a mutant mKO-FM14 which changes the fluorescence characteristic from green to orange. This mutant changes the fluorescence characteristic from green to orange according to passage of time. This mKO-FM14 was split into two fragments, and the N-terminal molecule (the amino acid sequence from the 1^{st} to the 168^{th}) and the C-terminal molecule (the amino acid sequence from the 169^{th} to the 218^{th}) were produced, to which different proteins that interacted each other were genetically ligated respectively. When the proteins that interact each other bind together, the N-terminal molecule and C terminal molecule of mKO-FM14 bind each other accordingly, forming a chromophore in the molecule, and fluorescence is emitted, and the fluorescence characteristic was changed from green color to orange color. In particular, by using this method, the interaction (binding) between the target proteins and then the history thereafter can be measured either in vitro or in vivo. Leucine zipper which is actually known to interact was fused to the C ends of the N-terminal molecule and the C-terminal molecule of mKO-FM14 to analyze the interaction. Leucine zippers used were leucine zipper acidic (LZA) and leucine zipper basic (LZB). LZA has negative charge and LZB has positive charge, and LZA does not interact with LZA, and LZB does not interact with LZB, but LZA and LZB binds each other on one to one basis (FIG. 1).

In the present invention, a fluorescent protein capable of emitting fluorescence with different colors according to the passage of time is used by splitting into an N-terminal fragment and a C-terminal fragment.

The type of the fluorescent protein capable of emitting fluorescence with different colors according to the passage of time is not particularly limited, and for example, a desired protein may be obtained by introducing a mutation to GFP or its variants (for example CFP, YFP or the like), but a fluorescent protein having an amino acid sequence represented by SEQ ID NO: 2 (the amino acid sequence of fluorescent protein monomer Kusabira-Orange (mKO)) in which the 70^{th} amino acid, proline, is substituted with other amino acid is preferably used. Particular examples of such fluorescent proteins include any of the following fluorescent protein.
(1) a fluorescent protein of an amino acid sequence represented by any of SEQ ID NO: 4, 6, 8, 10 and 12; or
(2) a fluorescent protein of an amino acid sequence represented by any of SEQ ID NO: 4, 6, 8, 10 and 12 wherein one or several amino acid are deleted, substituted and/or added, which has fluorescence characteristic which is changed from green color to orange color according to passage of time.

As used herein, the range of “one or several” in “an amino acid sequence represented by any of SEQ ID NO: 4, 6, 8, 10 and 12, in which one or several amino acid are deleted, substituted and/or added” is not particularly limited, and is meant for example, from 1 to 20, preferably from 1 to 10, more preferably from 1 to 7, even more preferably from 1 to 5 and especially preferably from 1 to about 3.

Also, the method for introducing a desired mutation to a predetermined amino acid sequence is publicly known to a person skilled in the art. For example, a DNA having a mutation may be constructed by appropriately using a publicly known technique for example site specific mutagenesis, PCR using degenerate oligonucleotides, mutagens for cells including nucleic acids or exposure to radiation. Using this DNA, a protein with an amino acid sequence having a mutation may be obtained. Such publicly known techniques are described, for example, in Molecular Cloning: A laboratory Manual, 2^{nd} Ed., Cold Spring Harbor Laboratory, Cold Spring Harbor, N.Y., 1989 and Current Protocols in Molecular Biology, Supplement 138, John Wiley & Sons (1987-1997).

In the present invention, a fusion protein of an N-terminal fragment of a fluorescent protein capable of emitting fluorescence with different colors with passage of time and a first test protein, and a fusion protein of a C-terminal fragment of the fluorescent protein and a second test protein, are used. Further, the fluorescent protein to be split is preferably a monomer forming type mutant fluorescent protein rather than a multimer forming type. The multimer forming type may affect the interaction assay of the test protein which was fused due to aggregation.

A combination of 2 kinds of proteins which interact each other may be used as test proteins in the present invention. The 2 kinds of test proteins which interact with each other may be respectively designated as the first test protein and the second test protein. The first and second test proteins are fused with the N-terminal fragment and the C-terminal fragment, respectively, of the fluorescent protein capable of emitting fluorescence with different colors with passage of time.

The method for obtaining the fused protein used in the present invention is not particularly limited. It may be chemically synthesized protein or recombinant protein produced by the gene recombinant technology, and preferably recombinant protein.

For producing a recombinant protein, it is necessary to first obtain DNA encoding the protein. By designing suitable primers by using the nucleotide sequence and amino acid sequence information of SEQ ID NO: 1-12 in the sequence listing and carrying out PCRs using DNA fragments containing the genes of such fluorescent protein as templates, DNA fragments which encode the N-terminal fragment and the C-terminal fragment of such fluorescent protein may be produced. Similarly, DNA fragments encoding proteins to be fused are also obtained. Then, by ligating these DNA fragments sequentially by gene recombination technology, the DNA encoding the desired fusion protein may be obtained.

In the present invention, interaction between a first test protein and a second test protein is analyzed by: allowing a fusion protein of an N-terminal fragment of a fluorescent protein capable of emitting different color of fluorescence according to passage of time and the first test protein and another fusion protein of a C-terminal fragment of the fluorescent protein and the second test protein coexist; letting the first test protein and the second test protein interact; and detecting the change in the fluorescence due to the interaction.

For example, two kinds of fusion proteins may be made to co-exist by expressing respective DNAs obtained as described above which encode the two kinds of fusion proteins at the same time in a cell. Alternatively, the two kinds of fusion proteins may be produced beforehand and mixed to make the two kinds of produced fusion proteins coexist.

An expression vector may be used in the case where two kinds of fusion proteins are made to coexist in a cell by expressing respective DNAs encoding the two kinds of fusion proteins at the same time in a cell. In the expression vector, the DNA encoding the fusion protein is ligated functionally to the elements necessary for transcription (for example, promoter and the like). The promoter is a DNA sequence which has a transcriptional activity in a host cell, and is appropriately selected according to the species of the host.

Examples of a promoter which can operate in bacterial cells may include a Bacillus stearothermophilus maltogenic amylase gene promoter, a Bacillus licheniformis alpha-amylase gene promoter, a Bacillus amyloliquefaciens BAN amylase gene promoter, a Bacillus subtilis alkaline protease gene promoter, a Bacillus pumilus xylosidase gene promoter, P_{R} and P_{L} promoters of phage rhamda, and lac, trp and tac promoters of Escherichia coli.

Examples of a promoter which can operate in mammalian cells may include an SV40 promoter, an MT-1 (metallothionein gene) promoter, and an adenovirus-2 major late promoter. Examples of a promoter which can operate in insect cells may include a polyhedrin promoter, a P10 promoter, an Autographa californica polyhedrosis basic protein promoter, a baculovirus immediate-early gene 1 promoter, and a baculovirus 39K delayed-early gene promoter. Examples of a promoter which can be operate in yeast host cells may include promoters derived from yeast glycolytic genes, an alcohol dehydrogenase gene promoter, a TPI1 promoter, and an ADH2-4c promoter.

Examples of a promoter which can operate in filamentous cells may include an ADH3 promoter and a tpiA promoter.

A transformant is produced by introducing the recombinant expression vector having DNA encoding the fusion protein into a suitable host, and then interaction between the proteins in the host can be analyzed by observing the fluorescence in the transformant.

Any cell can be used as a host cell into which the recombinant expression vector is introduced, as long as the two kinds of the fusion protein can be expressed therein. Examples of such a cell may include bacteria, yeasts, fungal cells, and higher eukaryotic cells.

Examples of bacteria may include Gram-positive bacteria such as Bacillus or Streptomyces, and Gram-negative bacteria such as Escherichia coli. These bacteria may be transformed by the protoplast method or other known methods, using competent cells.

Examples of mammalian cells may include HEK 293 cells, HeLa cells, COS cells, BHK cells, CHL cells, and CHO cells. A method of transforming mammalian cells and expressing the introduced DNA sequence in the cells is also known. Examples of such a method may include the electroporation, the calcium phosphate method, and the lipofection method.

Examples of yeast cells may include those belonging to Saccharomyces or Shizosaccharomyces. Examples of such cells may include Saccharomyces cerevisiae and Saccharomyces kluyveri. Examples of a method of introducing a recombinant vector into yeast host cells may include the electroporation, the spheroplast method, and the lithium acetate method.

Examples of other fungal cells may include those belonging to Filamentous fungi such as Aspergillus, Neurospora, Fusarium or Trichoderma. Where Filamentous fungi are used as host cells, transformation can be carried out by incorporating DNA constructs into host chromosomes, so as to obtain recombinant host cells. Incorporation of DNA constructs into the host chromosomes is carried out by known methods, and such known methods may include homologous recombination and heterologous recombination.

Where insect cells are used as host cells, both a vector into which a recombinant gene is introduced and a baculovirus are co-introduced into insect cells, and a recombinant virus is obtained in the culture supernatant of the insect cells. Thereafter, insect cells are infected with the recombinant virus, so as to allow the cells to express proteins (described in, for example, Baculovirus Expression Vectors, A Laboratory Manual; and Current Protocols in Molecular Biology, Bio/Technology, 6, 47 (1988)).

The Autographa californica nuclear polyhedrosis virus, which is a virus infecting to insects belonging to Barathra brassicae, can be used as baculovirus.

Examples of insect cells used herein may include Sf9 and Sf21, which are Spodoptera frugiperda ovarian cells [Baculovirus Expression Vectors, A Laboratory Manual, W. H. Freeman & Company, New York, (1992)], and HiFive (manufactured by Invitrogen), which are Trichoplusia ni ovarian cells.

Examples of the method of co-introducing both a vector into which a recombinant gene has been introduced and the above baculovirus into insect cells to prepare a recombinant virus may include the calcium phosphate method and the lipofection method.

The above transformant is cultured in an appropriate nutritive medium under conditions enabling the introduced DNA to be expressed.

In the present invention, the first test protein and the second test protein are made to interact each other intracellularly or extracellularly as described above, and the interaction between the first test protein and the second test protein can be analyzed by detecting a change in fluorescence due to the interaction. For analyzing the interaction of the test proteins with each other in a cell, the fluorescence of transformant can be observed and analyzed.

The observation of fluorescence may be carried out using, for example, a fluorescent microscope, an image analysis device and the like. The type of microscope may be chosen appropriately according to the purpose. For frequent observations such as monitoring of the change in time and the like, it is preferable to use a normal type epi-illumination fluorescent microscope. For better resolution to investigate the intracellular localization and the like in detail, a confocal laser microscope is preferable. From the viewpoint of keeping physiological conditions of cells and to prevent contamination, an inverted type microscope is preferable as the microscope system. On using an upright type microscope, a water immersion lens may be used as a high magnification lens. Further, a filter set may be chosen appropriately according to the wave length of fluorescence of the fluorescent protein used. Also, in the observation of living cells at various time points using a fluorescent microscope, a high sensitivity cooled CCD camera is used because pictures should be taken in a short time. The cooled CCD camera can take sharp pictures of weak fluorescent images with a short exposure time by reducing thermal noise by cooling CCD.

The present invention will be described more specifically by the following Examples, but is not limited thereby.

### EXAMPLES

### Example 1

### Preparation of mKO Mutant by Point Mutagenesis in which Multimer Formation is Blocked

The multimer formation boundary was predicted from the amino acid sequence of KO-1, and amino acids in the multimer formation boundary were substituted so that KO-1 was monomerized but the fluorescence characteristic was maintained. The introduction of the point mutation was carried out in an E. coli expression vector with KO-1 inserted thereinto (pRSET B) (the expression vector containing DNA encoding KO-1 described in International Publication WO03/54191) using primers for point mutagenesis. In particular, a multiplicity of point mutagenesis primers were annealed at the same time with one chain of the template plasmid and extended with polymerase. The DNA fragments extended by each primer were ligated in the same reaction mixture using DNA ligase. In this procedure, the DNA produced was complementary to the template except where the mutation was introduced. Since the ends of the DNA must be phosphorylated for ligating each DNA fragment by DNA ligase, the primers used were phosphorylated at the 5′ ends.

(1) Phosphorylation of 5′ Ends of Primers

| | | |
|---|---|---|
| 100 μM primer | 2 | μl |
| 10 × T4 polynucleotide kinase buffer | 5 | μl |
| 100 μM ATP | 0.5 | μl |
| Sterilized water | 41.5 | μl |
| T4 polynucelotide kinase (10 U/μl) | 1 | μl |

The above mixture was incubated at 37° C. for 30 minutes. The primers used herein have nucleotide sequences described in the SEQ ID NO: 29 to 43 below.

| | |
|---|---|
| K11R, F13Y | |
| CCAGAGATGAAGATGAGGTACTACATGGACGGC | (SEQ ID NO: 29) |
| V25I | |
| CATGAGTTCACAATTGAAGGTGAAGGC | (SEQ ID NO: 30) |
| K32R | |
| GAAGGCACAGGCAGACCTTACGAGGGA | (SEQ ID NO: 31) |
| S55A | |
| CCAATGCCTTTCGCGTTTGACTTAGTG | (SEQ ID NO: 32) |
| T62V | |
| TTAGTGTCACACGTGTTCTGTTACGGC | (SEQ ID NO: 33) |
| Q96E | |
| GAAAGGTCGTTGGAGTTCGAAGATGGT | (SEQ ID NO: 34) |
| F102S, A104S | |
| GAAGATGGTGGGTCCGCTTCAGTCAGTGCG | (SEQ ID NO: 35) |
| C115T, E117Y | |
| AGCCTTAGAGGAAACACCTTCTACCACAAATCCA | (SEQ ID NO: 36) |
| V123T | |
| CAAATCCAAATTTACTGGGGTTAACTTTCCTG | (SEQ ID NO: 37) |
| V133I | |
| GCCGATGGTCCTATCATGCAAAACCAAAGT | (SEQ ID NO: 38) |
| S139V | |
| GCCGATGGTCCTATCATGCAAAACCAAAGTGTTG | (SEQ ID NO: 39) |
| ATTGGGAGCCA | |
| T150A, C151S | |
| GAGAAAATTACTGCCAGCGACGGAGTTCTGAAG | (SEQ ID NO: 40) |
| F162Y, A166E | |
| GATGTTACGATGTACCTAAAACTTGAAGGAGGCG | (SEQ ID NO: 41) |
| GCAATCAC | |
| Q190G, F193Y, G195S | |
| CTTAAAATGCCAGGAAGCCATTACATCAGCCATC | (SEQ ID NO: 42) |
| GCCTCGTCAGG | |
| C217S | |
| GATGCAGTAGCTCATTCCCTCGAGCACCACCACC | (SEQ ID NO: 43) |

(2) Point Mutagenesis PCR

| | | |
|---|---|---|
| 5′phosphorylated primer | 4 | μl |
| template (KO-pRSET B) | 100 | ng |
| 10 × polymerase buffer | 2.5 | μl |
| 10 × DNA ligase buffer | 2.5 | μl |
| 2.5 mM dNTPs | 1 | μl |
| polymerase (pfu) 2.5 U/μl | 1 | μl |
| Taq DNA ligase 40 U/μl | 0.5 | μl |

Bring up to a total of 50 μl with sterilized water
Program

A thermal cycler GeneAmp PCR system 9700 was used.
1) 65° C. 5 min
2) 95° C. 2 min
3) 95° C. 20 sec
4) 52° C. 20 sec
5) 65° C. 8 min

Repeat 25 cycles above steps 3) to 5)
6) 75° C. 7 min
7) 4° C. hold
   (3) Dpn1 Treatment

The template plasmid was cleaved by adding 1 μl of Dpn1 to the sample after the PCR and incubating at 37° C. for 1 hour.

(4) Transfection to E. coli

The sample after the Dpn1 treatment was transfected to E. coli JM109 to express KO-1 after the mutagenesis.

(5) Amino Acid Sequence of Monomerized Kusabira-Orange (mKO)

The amino acid sequence was determined by analyzing the nucleotide sequence of KO mutant after the mutagenesis. Results indicate that the following substitutions were found: the 11^{th} lysine (K) was substituted with arginine (R); the 13^{th} phenylalanine (F) with tyrosine (Y); the 25^{th} valine (V) with isoleucine (I); the 32^{nd} lysine (K) with arginine (R); the 55^{th} serine (S) with alanine (A), the 62^{nd} threonine (T) with valine (V); the 96^{th} glutamine (Q) with glutamic acid (E); the 102^{nd} phenylalanine (F) with serine (S); the 104^{th} alanine (A) with serine (S); the 115^{th} cysteine (C) with threonine (T); the 117^{th} glutamic acid (E) with tyrosine (Y); the 123^{rd} valine (V) with threonine (T); the 133^{rd} valine (V) with isoleucine (I); the 139^{th} serine (S) with valine (V); the 150^{th} threonine (T) with alanine (A); the 151^{st} cysteine (C) with serine (S); the 162^{nd} phenylalanine (F) with tyrosine (Y); the 166^{th} alanine (A) with glutamic acid (E); the 190^{th} glutamine (Q) with glycine (G); the 193^{rd} phenylalanine (F) with tyrosine (Y); the 195^{th} glycine (G) with serine (S); and the 217 the cysteine (C) with serine (S). Further, to add the Kozak sequence, valine (V) was introduced in front of the 2^{nd} serine (S). This mutant was designated as mKO. The nucleotide sequence of mKO is shown in SEQ ID NO: 1 in the sequence listing, and the amino acid sequence is shown in SEQ ID NO: 2 in the sequence listing.

mKO protein added with a His-Tag was expressed in E. coli by a conventional method, and was purified using Ni-Agarose.

### Example 2

### Analysis of Fluorescence Characteristic

Fluorescent and absorption spectra of mKO protein purified in Example 1 were measured as follows, and quantum yield and molar absorption coefficient were calculated.

Absorption spectra were measured using the 20 μM fluorescent protein, 50 mM HEPES pH 7.5 solution. The molar absorption coefficient was calculated from the peak values of the spectra. The absorption peak was observed at 548 nm for mKO. The fluorescent protein was diluted with the aforementioned buffer so that the absorbance at 500 nm was 0.0025, and the fluorescence spectra with excitation by 500 nm and excitation spectra by fluorescence at 590 nm were measured. DsRed (CLONTECH) was similarly diluted so that the absorbance at 500 nm was 0.0025, and the fluorescent spectra were measured and the quantum yield of mKO was obtained assuming the quantum yield of DsRed was 0.29.

The results are shown in Table 1. The data of KO protein (dimer protein) described in International Publication WO03/54191 are also shown in Table 1.

TABLE 1

| | | | Molar | | Amino | | |
|---|---|---|---|---|---|---|---|
| | Excitation | Fluorescence | absorption | Quantum | acid | Multimer | pH |
| | peak | peak | coefficient | yield | number | formation | sensitivity |
| KO | 548 nm | 561 nm | 109750 | 0.45 | 217 | Dimer | pKa < 5.0 |
| mKO | 548 nm | 559 nm | 51600 | 0.6 | 218 | Monomer | pKa = 5.0 |

### Example 3

### Measurement of Molecular Weight by Ultracentrifugation Analysis

mKO protein solution was prepared in 150 mM KCl, 50 mM HEPES-KOH, pH 7.4, and subjected to ultracentrifugation analysis to determine the molecular weight of mKO. The solution was centrifuged using an ultracentrifuge XL-1 (Beckman Coulter) at 25,000 rpm for 22 hours, and absorbance at 540 nm, which was close to the absorption peak (548 nm) of mKO, was measured. The molecular weight of mKO was calculated to be 28 kDa from the result of the measurement. This value is almost the same with 26 kDa which is predicted from the amino acid sequence, confirming that mKO exists as monomer.

### Example 4

### Preparation of mKO Variant which Emits 2 Fluorescence, Green and Orange (Passage of Time Measurement Probe and Monitoring Probe)

Fluorescent proteins having different fluorescence characteristics than mKO were produced by substituting amino acids of mKO. mKO emits green fluorescence immediately after the translation and then emits orange fluorescence after that. However, transition from green fluorescence to orange fluorescence is completed so fast that normally it cannot be observed. Therefore, fluorescent proteins having various ratios of green fluorescence and orange fluorescence according to the passage of time were produced. By using these variants, the time from the protein expression can be measured by the ratio of green fluorescence and orange fluorescence. Further, in these variants, green fluorescence and orange fluorescence were independent, and thus it was possible to extinguish orange fluorescence only. In particular, reset of the measurement of the passage of time becomes possible by extinguishing orange fluorescence only, and then measuring the increase of orange fluorescence. Similarly, by extinguishing only an optional part of orange fluorescence and by measuring the ratio of green fluorescence and orange fluorescence, the behavior of molecules and cells labeled with the extinguished part can be measured. The results revealed that by substituting the 70^{th} proline (P) with other amino acid, fluorescent proteins can be produced that emit fluorescence with a variety of ratios of green fluorescence and orange fluorescence according to the passage of time.

(1) Mutagenesis

By substituting amino acids of mKO, fluorescent proteins having different fluorescence characteristic than mKO were produced. The point mutagenesis was carried out by PCR of an E. coli expression vector (pRSET_{B}) into which mKO was inserted, using primers for point mutagenesis. The primers used for PCR were phosphorylated at the 5′ ends.

(a) Phosphorylation of 5′ End of Primers

| | | |
|---|---|---|
| 100 μM primer | 2 | μl |
| 10 × T4 polynucleotide kinase buffer | 5 | μl |
| 100 μM ATP | 0.5 | μl |
| Sterilized water | 41.5 | μl |
| T4 polynucleotide kinase (10 U/μl) | 1 | μl |

The reaction mixture was incubated at 37° C. for 30 min.

(b) Point Mutagenesis PCR

| | | |
|---|---|---|
| 5′phosphorylated primer | 4 | μl |
| template (mKO-pRSET_{B}) | 100 | ng |
| 10 × polymerase buffer | 2.5 | μl |
| 10 × DNA ligase buffer | 2.5 | μl |
| 2.5 mM dNTPs | 1 | μl |
| polymerase (pfu) 2.5 U/μl | 1 | μl |
| Taq DNA ligase 40 U/μl | 0.5 | μl |

Bring up to a total of 50 μl with sterilized water
Program

A thermal cycler GeneAmp PCR system 9700 was used.
1) 65° C. 5 min
2) 95° C. 2 min
3) 95° C. 20 sec
4) 52° C. 20 sec
5) 65° C. 8 min
6) 75° C. 7 min
7) 4° C. hold

Repeat 25 cycles above steps 3) to 5)

(c) Dpn1 Treatment

The template plasmid was cleaved by adding 1 μl of Dpn1 to the sample after the PCR and incubating at 37° C. for 1 hour.

(d) Transfection to E. coli

The sample after the Dpn1 treatment was transfected to E. coli JM109 (DE3) to express mKO after the mutagenesis for analysis.

(2) Analysis of mKO Time Passage Variant

The nucleotide sequence analysis of a produced variant of mKO revealed that the 49^{th} lysine (K) was substituted with glutamic acid (E); the 70^{th} proline (P) with glycine (G); the 185^{th} lysine (K) with glutamic acid (E); the 188^{th} lysine (K) with glutamic acid (E); the 192^{nd} serine (S) with aspartic acid (D); the 196^{th} serine (S) with glycine (G). This mKO variant was a fluorescent protein which emitted fluorescence with different ratios of green fluorescence and orange fluorescence according to the passage of time. The change rate of the ratios of green fluorescence and orange fluorescence according to the passage of time was varied by substituting the 70^{th} proline (P) of this mKO variant with various other amino acids.

The variant substituted with valine (V) was designated as mKO-FM14 (the amino acid sequence is shown in SEQ ID NO: 4 and the nucleotide sequence is shown in SEQ ID NO: 3).

The variant substituted with alanine (A) was designated as mKO-FM5 (the amino acid sequence is shown in SEQ ID NO: 6 and the nucleotide sequence is shown in SEQ ID NO: 5).

The variant substituted with serine (S) was designated as mKO-FM3 (the amino acid sequence is shown in SEQ ID NO: 8 and the base sequence is shown in SEQ ID NO: 7).

The variant substituted with cysteine (C) was designated as mKO-FM20 (the amino acid sequence is shown in SEQ ID NO: 10 and the base sequence is shown in SEQ ID NO: 9).

The variant substituted with threonine (T) was designated as mKO-FM24 (the amino acid sequence is shown in SEQ ID NO: 12 and the base sequence is shown in SEQ ID NO: 11).

Measurement of each time passage variant mKO was carried out for recombinant fluorescent protein either expressed in E. coli JM109 (DE3), or in vitro translation system PURE SYSTEM CLASSIC MINI (Post Genome Institute). In the measurement in E. coli, culture plates in which each variant was expressed were incubated at 37° C. and the excitation spectra at 580 nm were measured by sampling at various time intervals. The results indicated that, compared to the about 500 nm peak of the excitation peak of green fluorescence, the 548 nm peak of the excitation peak of orange fluorescence was increased with time and the rate of increase was different in each variant. The peak of green fluorescence was 509 nm and that of orange fluorescence was 560 nm. Fluorescence measurement was carried out using a spectrofluorometer F-2500 (HITACHI). Since new protein is produced discontinuously in E. coli, the apparent time required for transition from green to orange becomes longer. Therefore, the production time of protein was limited by using in vitro translation system and more accurate measurement for transition from green to orange according to the passage of time was carried out. The time for protein synthesis was fixed to be 1 hour. Immediately after that, energy sources required for protein synthesis, such as ATP and the like, were removed by gel filtration, and the mixture was incubated at 37° C., and excitation spectra at 580 nm were measured up to 25 hours after the synthesis.

### Example 5

### Construction

N-terminal fragment from the 1^{st} to the 168^{th} of mKO-FM14 amino acid sequence(mKO-FM14-N) and C-terminal fragment from 169^{th} to 218^{th} (mKO-FM14-C) were separately amplified by PCR using mKO-FM14 as a template. A primer was designed so that the translation initiation methionine (M) was added in front of 169^{th} glycine (G) of C-terminal fragment.

Primer for Amplification of N-Terminal Fragment (1^{st}-168^{th}) of mKO-FM14

| | | |
|---|---|---|
| AAAAAGCTTACCATGGTGAGTGTG | (primer1) | (SEQ ID NO: 44) |
| ATTAAACCAGAGATG | | |
| TGCAGAATTCCCTCCTTCAAGTTT | (primer2) | (SEQ ID NO: 45) |
| TAGGTACATCGT | | |

Primer for Amplification of C-Terminal Fragment (169^{th}-218^{th}) of mKO-FM14

| | | |
|---|---|---|
| AAAAAGCTTACCATGGGCGGCAAT | (primer3) | (SEQ ID NO: 46) |
| CACAAATGCCAATTC | | |
| TGCAGAATTCCCGGAATGAGCTAC | (primer4) | (SEQ ID NO: 47) |
| TGCATCTTC | | |

Composition of PCR Reaction Mixture

| | | |
|---|---|---|
| Template (mKO-FM14) | 1 | μl |
| X 10 pfu buffer | 5 | μl |
| 2.5 mM dNTPs | 4 | μl |
| 20 μM primer 1 or 3 | 1 | μl |
| 20 μM primer 2 or 4 | 1 | μl |
| Milli Q | 37 | μl |
| DMSO | 5 | μl |
| Pfu DNA polymerase (5 U/μl) | 1 | μl |

PCR Reaction Condition
94° C. 1 min (PAD)
94° C. 30 sec (Denature)
52° C. 30 sec (Annealing of primer to template)
72° C. 1 min (Extension of primer)
72° C. 7 min (Last extension)
4° C. Holding

The amplification product was separated by agarose gel electrophoresis, excised out and purified. The fragment was cleaved by restriction enzymes, HindIII and EcoRI, purified and used for the construction.

LZA (amino acid sequence: RAQLEKELQALEKENAQLEWELQALEKELAQK) (SEQ ID NO: 48) and LZB (amino acid sequence: RAQLKKKLQALKKKNAQLKWKLQALKKKLAQK) (SEQ ID NO: 49) were prepared by DNA synthesis. mKO-FM14-N and LZA (mKO-FM14-N-LZA), and mKO-FM14-C and LZB (mKO-FM14-C-LZB) were fused genetically. A 26 amino acid sequence of GNSADGGGGSGGGGSGGGGSIHHTGG (SEQ ID NO: 50) was inserted to the fusion site as a linker. mKO-FM14-N-LZA and mKO-FM14-C-LZB were subcloned separately to the HindIII-XhoI sites of expression vector pCDNA3 (Invitrogen) and used in the following experiments (mKO-FM14-N-LZA-pCDNA3 and mKO-FM14-C-LZB-pCDNA3).

### Example 6

### Protein Synthesis by in vitro Translation

To analyze the interaction between mKO-FM14-N-LZA and mKO-FM14-C-LZB, proteins were synthesized by in vitro translation. For protein synthesis by in vitro translation, PureSystem (Postgenome Institute Inc.) was used. Following the instruction of the kit, PCR amplification was carried out twice and the product was used as a template for protein synthesis for in vitro translation.

Amplification Primer for mKO-FM14-N-LZA for the First PCR

| | | |
|---|---|---|
| AAGGAGATATACCAATGGTGAGTG | (primer5) | (SEQ ID NO: 51) |
| TGATTAAACCAGAG | | |
| TATTCATTACTTCTGGGCCAG | (primer6) | (SEQ ID NO: 52) |

Amplification Primer for mKO-FM14-C-LZB for the First PCR

| | | |
|---|---|---|
| AAGGAGATATACCAATGGGCAATC | (primer7) | (SEQ ID NO: 53) |
| ACAAATGCCAATTC | | |
| TATTCATTACTTCTGGGCCAG | (primer6) | (SEQ ID NO: 52) |

Composition of the First PCR Reaction Mixture

Template (mKO-FM14-N-LZA-pCDNA3 or mKO-FM14-C-LZB-pCDNA3) 1 μl

| | | |
|---|---|---|
| X 10 pfu buffer | 5 | μl |
| 2.5 mM dNTPs | 4 | μl |
| 2 μM primer 5 or 7 | 1 | μl |
| 2 μM primer 6 | 1 | μl |
| Milli Q | 32 | μl |
| DMSO | 5 | μl |
| pfu DNA polymerase (5 U/μl) | 1 | μl |

PCR Reaction Condition
94° C. 1 min (PAD)
94° C. 30 sec (Denature)
42° C. 30 sec (Annealing of primer to template)
72° C. 1 min (Extension of primer)
72° C. 7 min (Last extension)
4° C. Holding

Each amplification product was diluted by 50 fold, and the second PCR was carried out using this as a template.

Amplification Primer for mKO-FM14-N-LZA for the Second PCR

| | | |
|---|---|---|
| GAAATTAATACGACTCACTATA | (primer8: | (SEQ ID NO: 54) |
| GGGAGACCACAACGGTTTCCCT | included in | |
| CTAGAAATAATTTTGTTTAACT | the kit) | |
| TTAAGAAGGAGATATACCA | | |
| TATTCATTACTTCTGGGCCAG | (primer6) | (SEQ ID NO: 52) |

Amplification Primer for mKO-FM14-C-LZB for the Second PCR

| | | |
|---|---|---|
| GAAATTAATACGACTCACTATA | (primer8: | (SEQ ID NO: 54) |
| GGGAGACCACAACGGTTTCCCT | included in | |
| CTAGAAATAATTTTGTTTAACT | the kit) | |
| TTAAGAAGGAGATATACCA | | |
| TATTCATTACTTCTGGGCCAG | (primer6) | (SEQ ID NO: 52) |

Composition of the Second PCR Reaction Mixture

Template (the first PCR amplification product was diluted by 50 fold) 1 μl

| | | |
|---|---|---|
| X 10 taq buffer | 5 | μl |
| 2.5 mM dNTPs | 4 | μl |
| 2 μM primer 8 | 1 | μl |
| 2 μM primer 6 | 1 | μl |
| Milli Q | 37 | μl |
| taq DNA polymerase (5 U/μl) | 1 | μl |

PCR Reaction Condition
94° C. 1 min (PAD)
94° C. 30 sec (Denature)
42° C. 30 sec (Annealing of primer to template)
72° C. 1 min (Extension of primer)
72° C. 7 min (Last extension)
4° C. Holding

The second PCR amplification product (5 μl) was used for one in vitro translation. The in vitro translation was carried out according to the method of the kit. Protein was synthesized at 37° C. for 1 hour and used for the measurement.

As negative controls, templates for in vitro translation of mKO-FM14-N and mKO-FM14-C, which did not contain leucine zipper, were similarly prepared. As PCR templates, mKO-FM14-N-LZA-pCDNA3 and mKO-FM14-C-LZB-pCDNA3 were used. For each template, reverse primers were designed in which a stop codon was inserted before a linker so that the linker and leucine-zipper were not translated, and PCR was carried out twice as described above. As the result, the PCR amplification products of mKO-FM14-N and mKO-FM14-C, which did not contain leucine zipper, were obtained. This PCR amplification product (5 μl) was used for one in vitro translation.

Amplification Primer for mKO-FM14-N for the First PCR

| | | |
|---|---|---|
| AAGGAGATATACCAATGGTGAGTG | (primer5) | (SEQ ID NO: 51) |
| TGATTAAACCAGAG | | |
| TATTCATTATCCTTCAAGTTTTAG | (primer9) | (SEQ ID NO: 55) |
| GTACAT | | |

Amplification Primer for mKO-FM14-C for the First PCR

| | | |
|---|---|---|
| AAGGAGATATACCAATGGGCAA | (primer7) | (SEQ ID NO: 53) |
| TCACAAATGCCAATTC | | |
| TATTCATTAGGAATGAGCTACT | (primer10) | (SEQ ID NO: 56) |
| GCATCTTCTACCA | | |

Amplification Primer for mKO-FM14-N for the Second PCR

| | | |
|---|---|---|
| GAAATTAATACGACTCACTATA | (primer8: | (SEQ ID NO: 54) |
| GGGAGACCACAACGGTTTCCCT | included in | |
| CTAGAAATAATTTTGTTTAACT | the kit) | |
| TTAAGAAGGAGATATACCA | | |
| TATTCATTATCCTTCAAGTTTT | (primer9) | (SEQ ID NO: 55) |
| AGGTACAT | | |

Amplification Primer for mKO-FM14-C for the Second PCR

(SEQ ID NO: 54)

GAAATTAATACGACTCACTATAGGGAGACCACAACGGTTTCCCTCTAGAAA
TAATTTTGTTTAACTTTAAGAAGGAGATATACCA
(primer8: included in the kit)

(SEQ ID NO: 53)

AAGGAGATATACCAATGGGCAATCACAAATGCCAATTC
(primer7)

(SEQ ID NO: 56)

TATTCATTAGGAATGAGCTACTGCATCTTCTACCA
(primer10)

### Example 7

### Analysis of Interaction between mKO-FM14-N-LZA and mKO-FM14-C-LZB

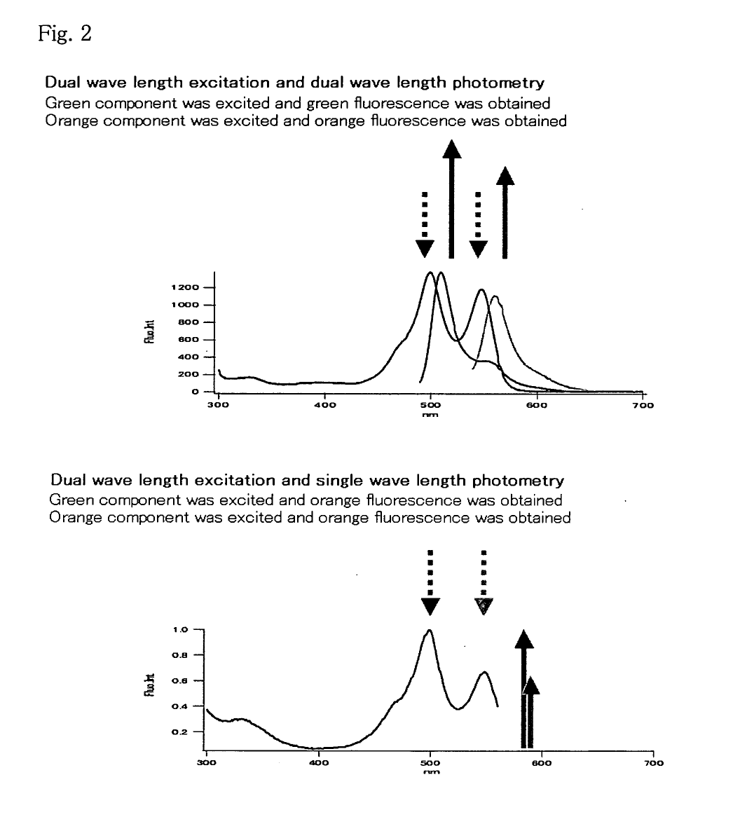

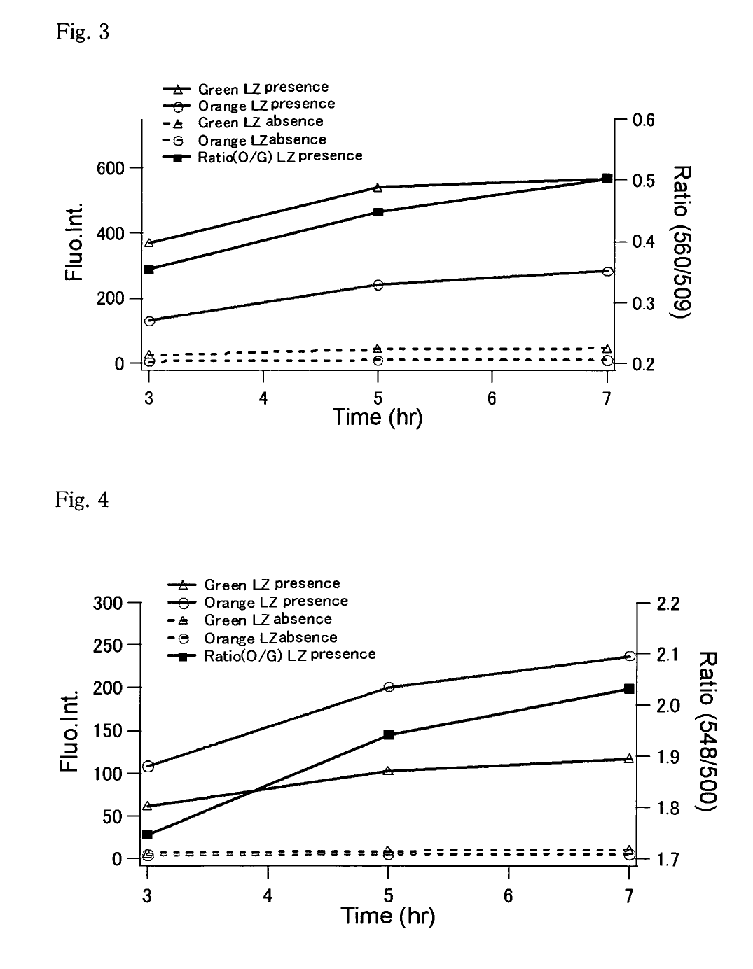

20 μl of each mKO-FM14-N-LZA and mKO-FM14-C-LZB which was synthesized by in vitro translation was added to 1000 μl of PBS and the total volume was made up to 1040 μl. The mixture was left standing at 37° C. and the fluorescence measurements were carried out every 2 hours (after 3, 5 and 7 hours from the mixing) by a F-2500 (HITACHI). The method of measurement was by the double wavelength excitation and a single wavelength measurement (excitation spectra at 580 nm, green component and orange component were set at 500 nm and 548 nm, respectively) or by the double wavelength excitation and double wavelength measurement (green fluorescence at 509 nm by excitation by 470 nm and orange fluorescence at 560 nm by excitation by 520 nm were obtained) (FIG. 2). Also, as negative controls, 20 μl of each mKO-FM14-N and mKO-FM14-C which was synthesized by in vitro translation and to which no proteins for interaction were fused was added to 1000 μl of PBS and the total volume was made up to 1040 μl. The mixture was left standing at 37° C. and the fluorescence measurements were carried out every 2 hours (after 3, 5 and 7 hours from the mixing) by a F-2500 (HITACHI). As a result, in the mixture of mKO-FM14-N-LZA and mKO-FM14-C-LZB left standing, an increase of fluorescence as well as an increase of the ratio of orange/green with time were observed, and no increase of fluorescence was observed in mKO-FM14-N and mKO-FM14-C (FIG. 3, 4), and thus the maturation process of fluorescent protein mKO-FM14 from green to orange, which was dependent on the binding (interaction) of LZA and LZB, was observed.

### Example 8

### Analysis of Interaction between LZA and LZB by mKO Variants having Different Maturation Time

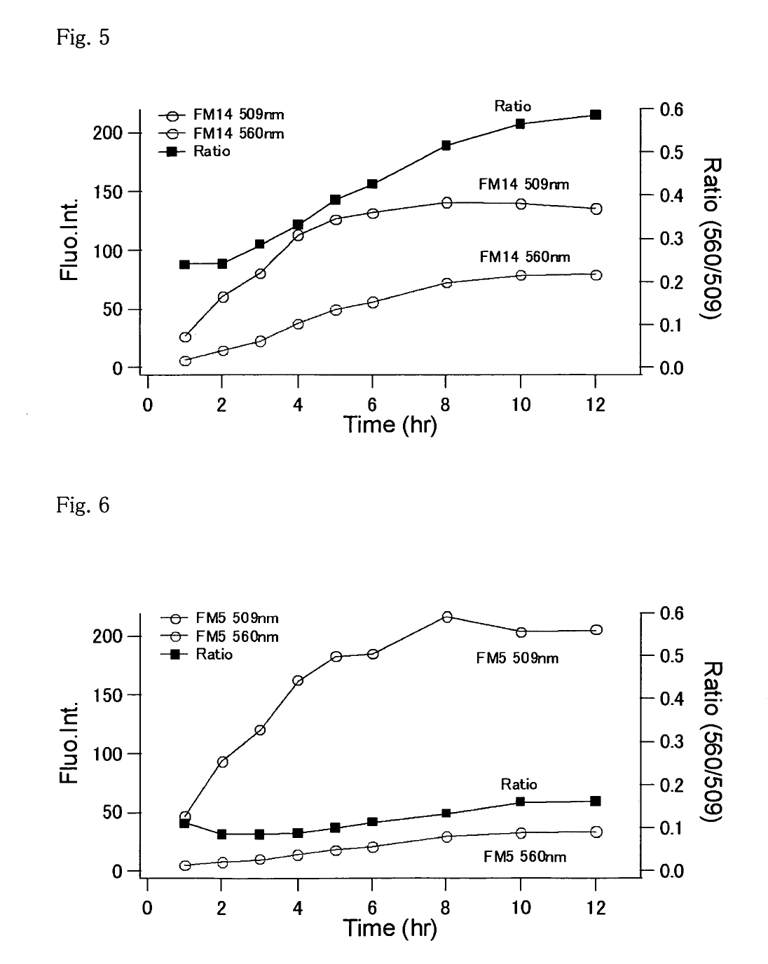

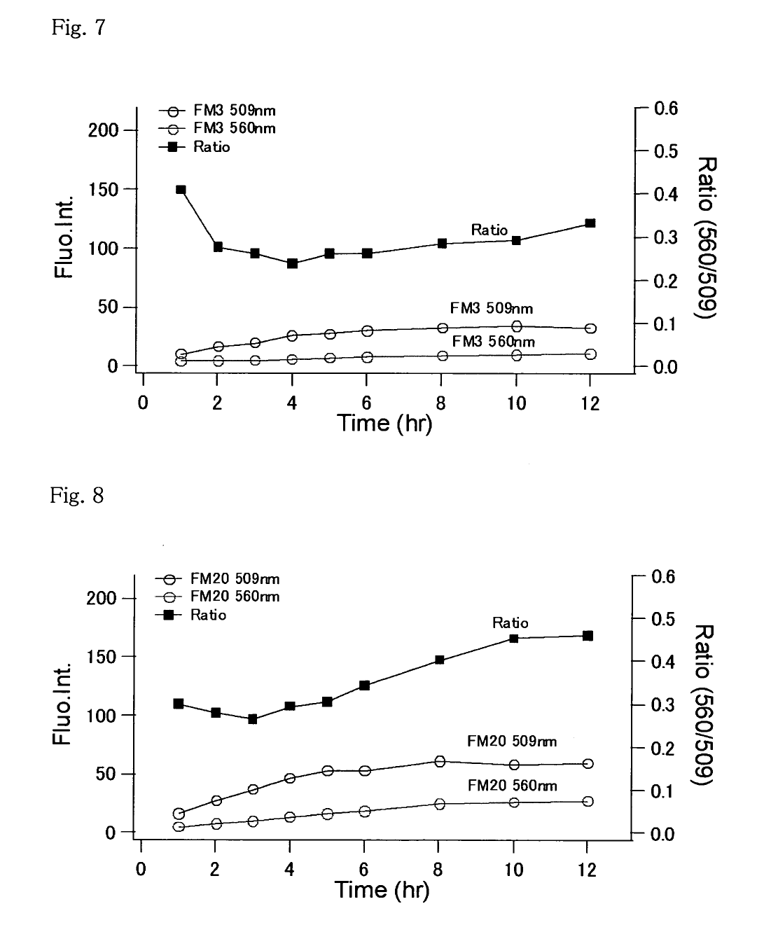

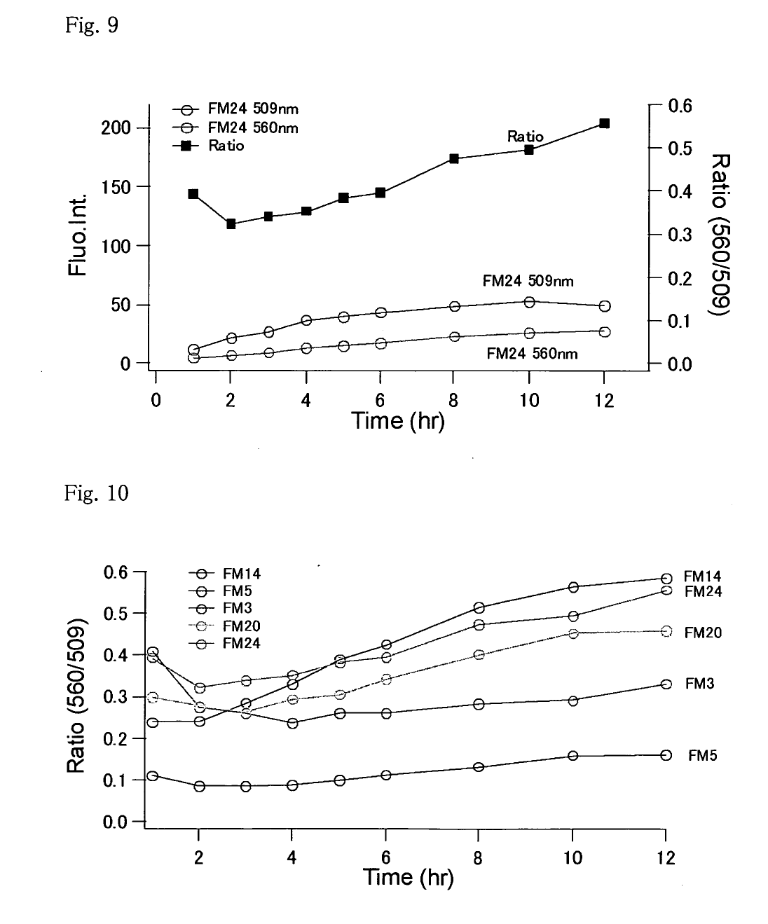

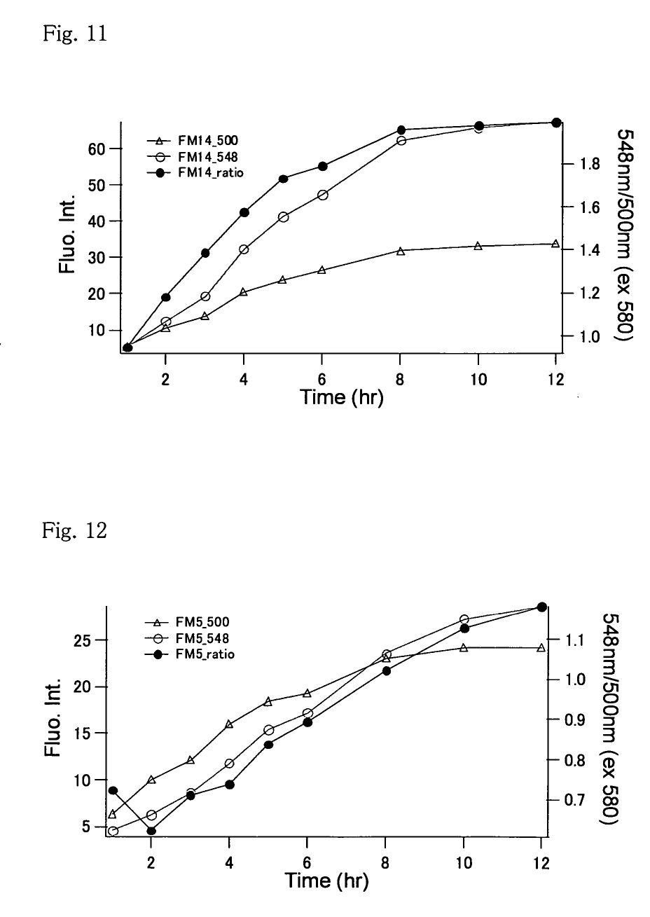

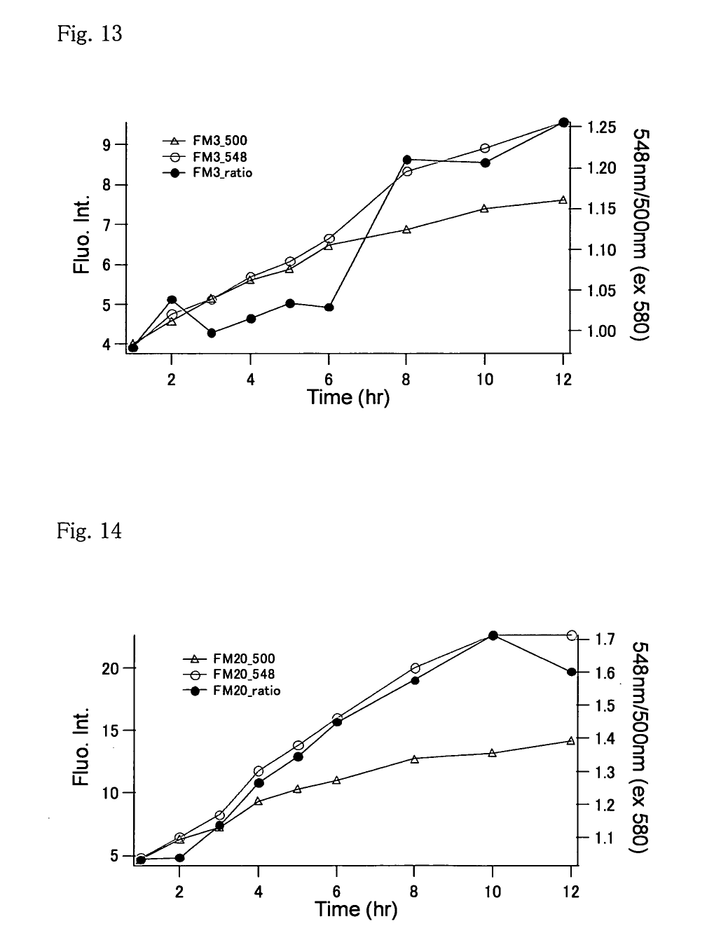

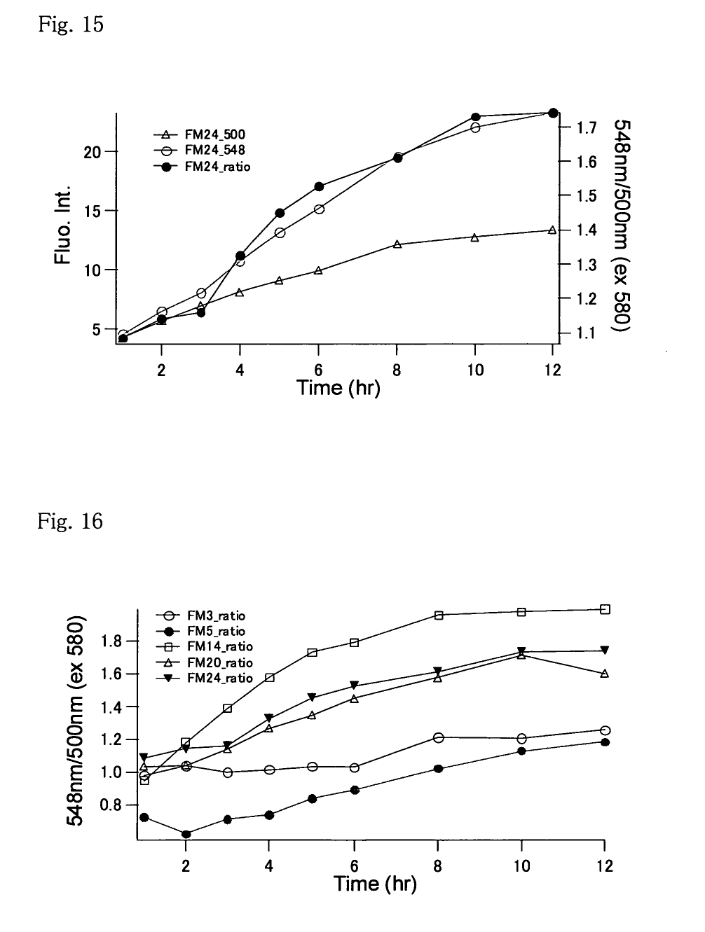

By substituting the 70^{th} proline (P) of mKO with various amino acids, variants were obtained in which the ratio of green fluorescence and orange fluorescence was changed according to the passage of time. The variant in which 70^{th} proline (P) was substituted with alanine (A), serine (S), cysteine (C), threonine (T), and valine (V) was designated as mKO-FM5, mKO-FM3, mKO-FM20, mKO-FM24 and mKO-FM14, respectively. Each variant was similarly split into two molecules and similarly fused with LZA and LZB to carry out analyses. Since the site of the variation was the 70^{th} amino acid, only the fusion products of N-terminal fragment (from 1^{st} to 168^{th}) of each variant and LZA were genetically produced, and the mKO-FM14-C-LZB was used for the measurement as the C-terminal fragment (from 169^{th} to 218^{th}), which did not influence the characteristics of the variants. The fluorescence measurement was carried out according to the method described above and from 1 hour after mixing and up to 12 hour. As the result, in these variants, the maturation process of fluorescent protein from green to orange, which was dependent on the binding (interaction) of LZA and LZB, was also observed. Further, there were variations in fluorescence intensity and degree of maturation (time) among these variants, and it was thought that these variants may be used differently depending on the objective (FIG. 5,6,7,8,9,10,11,12,13,14,15 and 16).

The nucleotide sequences and amino acid sequences of the constructs used in the above Examples are shown in the sequence listing as mentioned below.
Nucleotide sequence of mKO-FM14-N-LZA (SEQ ID NO: 13)
Amino acid sequence of mKO-FM14-N-LZA (SEQ ID NO: 14)
Nucleotide sequence of mKO-FM14-C-LZB (SEQ ID NO: 15)
Amino acid sequence of mKO-FM14-C-LZB (SEQ ID NO: 16)
Nucleotide sequence of mKO-FM14-N (SEQ ID NO: 17)
Amino acid sequence of mKO-FM14-N (SEQ ID NO: 18)
Nucleotide sequence of mKO-FM14-C (SEQ ID NO: 19)
Amino acid sequence of mKO-FM14-C (SEQ ID NO: 20)
Nucleotide sequence of mKO-FM20-N-LZA (SEQ ID NO: 21)
Amino acid sequence of mKO-FM20-N-LZA (SEQ ID NO: 22)
Nucleotide sequence of mKO-FM3-N-LZA (SEQ ID NO: 23)
Amino acid sequence of mKO-FM3-N-LZA (SEQ ID NO: 24)
Nucleotide sequence of mKO-FM5-N-LZA (SEQ ID NO: 25)
Amino acid sequence of mKO-FM5-N-LZA (SEQ ID NO: 26)
Nucleotide sequence of mKO-FM24-N-LZA (SEQ ID NO: 27)
Amino acid sequence of mKO-FM24-N-LZA (SEQ ID NO: 28)

### Example 9

### Analysis of Intracellular Interaction between mKO-FM14-N-p21 and mKO-FM14-C-PCNA

The proteins which are known to interact with each other were fused to the C-terminal of mKO-FM14-N and the C-terminal of mKO-FM14-C, respectively and expressed in HeLa cells to measure the process after intracellular interaction.

The motives of p21 genetically linked to the C-terminal of mKO-FM14-N and PCNA genetically linked to the C-terminal of mKO-FM14-C were prepared. p21, which is also known as WAF1, Sdi1 or Cip1, is a protein consisting of total 164 amino acids. It has been known that expression of p21 is induced by p53 and had been isolated as a gene specifically highly-expressed in cells in advanced aging. PCNA (proliferating cell nuclear antigen) is a protein consisting of 261 amino acids, and is known to form a homo-trimer. PCNA is involved in DNA replication/repair and functions in the nucleus. It is believed that p21 affects DNA replication by binding with PCNA. p21 acts for stopping cell growth by binding PCNA or other cell cycle regulating factors.

mKO-FM14-N-LZA-pCDNA3 was cleaved with NotI and XhoI to remove LZA, and p21, to which the recognition sequences of NotI and XhoI were added by PCR, was inserted to construct mKO-FM14-N-p21-pCDNA3. Similarly, mKO-FM14-C-LZB-pCDNA3 was cleaved with NotI and XhoI to remove LZB, and PCNA, to which the recognition sequences of NotI and XhoI were added by PCR, was inserted to construct mKO-FM14-C-PCNA-pCDNA3.

Primer for p21 amplification

(SEQ ID NO: 63)

| | |
|---|---|
| ACTGGCGGCCGCATGTCAGAACCGGCTGGGGATGT | (primer11) |

(SEQ ID NO: 64)

| | |
|---|---|
| GGGCTCGAGTTAGGGCTTCCTCTTGGAGAAGAT | (primer12) |
| Primer for PCNA amplification | |

(SEQ ID NO: 65)

| | |
|---|---|
| ACTGGCGGCCGCATGTTCGAGGCGCGCCTGGTCCA | (primer13) |

(SEQ ID NO: 66)

| | |
|---|---|
| GGGCTCGAGTTAAGATCCTTCTTCATCCTCGATCTT | (primer14) |

Composition of PCR Reaction Mixture

| | | |
|---|---|---|
| Template (HeLa-cDNA library) | 1 | μl |
| X 10 pfu buffer | 5 | μl |
| 2.5 mM dNTPs | 4 | μl |
| 20 μM primer 11 or 13 | 1 | μl |
| 20 μM primer 12 or 14 | 1 | μl |
| Milli Q | 37 | μl |
| DMSO | 5 | μl |
| pfu DNA polymerase (5 U/μl) | 1 | μl |

PCR Reaction Condition
94° C. 1 min (PAD)
94° C. 30 sec (Denature)
52° C. 30 sec (Annealing of primer to template)
72° C. 1 min (Extension of primer)
72° C. 7 min (Last extension)
4° C. Holding

The amplification product was separated by agarose gel electrophoresis, excised out and purified and then cleaved with restriction enzymes NotI and XhoI, purified and used for the construction. mKO-FM14-N-p21-pCDNA3 and mKO-FM14-C-PCNA-pCDNA3 thus prepared were transfected to HeLa cells to obtain the history data of binding of p21 and PCNA. HeLa cells were grown in 3.5 cm dishes to 30% confluence, and after 16 hours, 500 ng each of the plasmid, mKO-FM14-N-p21-pCDNA3 and mKO-FM14-C-PCNA-pCDNA3, was transfected using the gene transfection reagent polyfect (QIAGEN) to co-express mKO-FM14-N-p21 and mKO-FM14-C-PCNA. Gene transfection was carried out according to the protocol of polyfect.

After 8 and 22 hours of the gene transfection, orange and green fluorescence images were obtained. However, “after 8 and 22 hours of the gene transfection” means “after 8 and 22 hours of the addition of the gene transfection reagent and expression plasmid complexes” and not “after 8 and 22 hours of the incorporation of the expression plasmids into the cells. Orange fluorescence image was obtained using the excitation filter 25BP520-540HQ, the fluorescence filter 25BA555-600HQ, and the dichroic mirror DM545HQ, and green fluorescence image was obtained using the excitation filter BP460-480, the fluorescence filter BA495-540, and the dichroic mirror DM485. Excitation light was cut by 97% (3% transmission), and the exposure time was 1 second. An inverted microscope IX-71 (Olympus) with 40× Uapo/340 N.A. 1.35 lens (Olympus) was used. For image retrieval and analysis, MetaMolph (Nippon Roper Inc.) was used by binning 2 to obtain an image. Fluorescence image was obtained using a cooling CCD camera ORCA-ER (Hamamatsu Photonics Inc.).

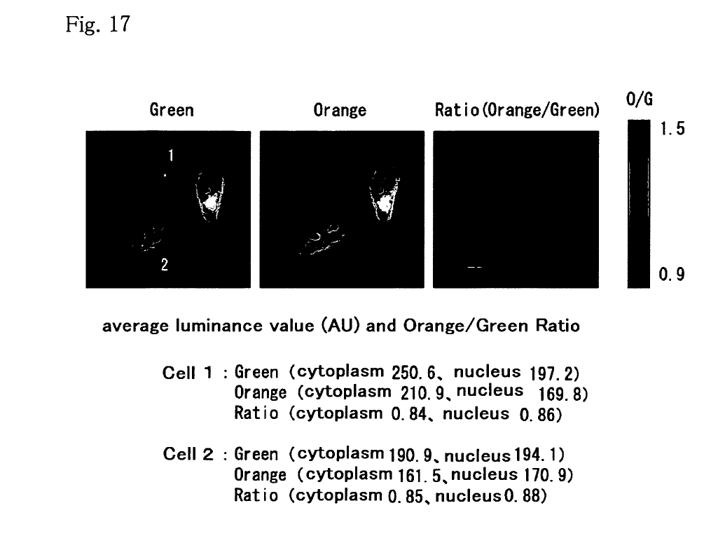

After 8 hours, there was a difference in luminance between the cytoplasm and nucleus, and in cell No. 1, the average luminance value was cytoplasm>nucleus for both of green and orange and in cell No. 2, it was cytoplasm<nucleus. The orange/green ratio (Ratio) was measured to be higher in nucleus than cytoplasm in both cells. This result shows that p21 and PCNA are moved to the nucleus after binding in the cytoplasm (FIG. 17). Assuming that p21 and PCNA act on DNA replication in the nucleus, it is assumed to be the reasonable result.

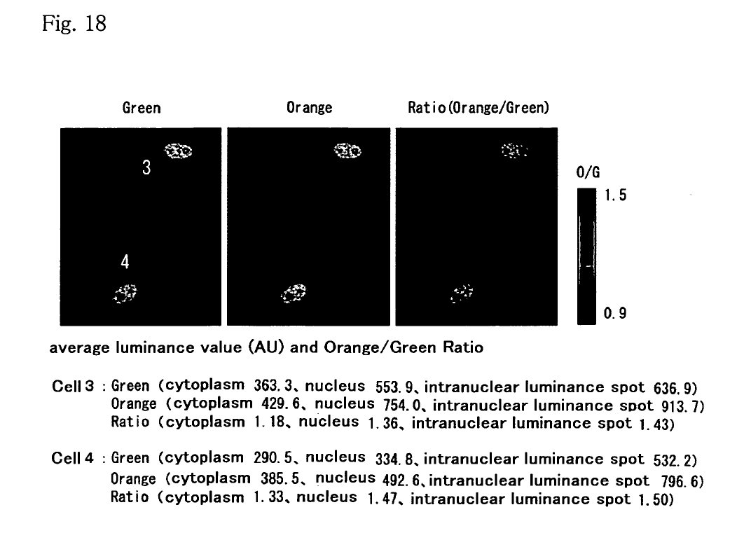

After 22 hours, in both cell No. 3 and cell No. 4, formation of clusters (intranuclear luminance spot) of p21 and PCNA complex were observed in the nucleus. The intranuclear clusters were thought to be the sites for DNA repair or DNA replication. Further the Ratio values were higher in the cytoplasm, nucleus and intranuclear luminance spot in this order. These results suggest that the Ratio image indicates that the complex of p21 and PCNA move from the cytoplasm to nucleus, and from the nucleus to the intranuclear luminance spots (FIG. 18).

### Comparative Example 1

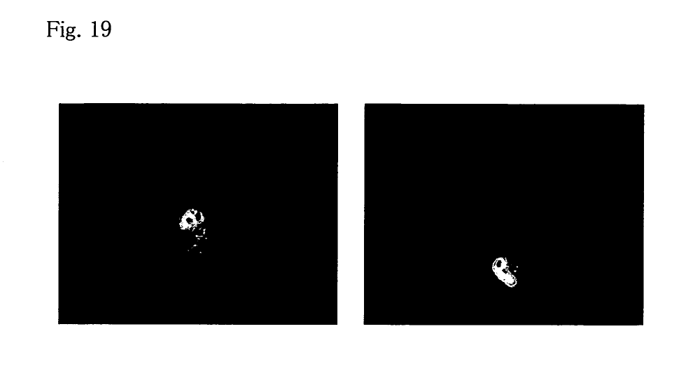

By substituting the 65^{th} cysteine (C) of mKO-FM14 with alanine (A), a green fluorescent protein (mKG), which did not have the timer function of green to orange shift, was obtained. Since the 65^{th} amino acid was included in mKO-FM14-N fragment, mKG-N-p21-pCDNA3 was produced by substituting the 65^{th} cystein (C) of mKO-FM14-N-p21-pCDNA3 with alanine (A). HeLa cells were transfected with mKG-N-p21-pCDNA3 and mKO-FM14-C-PCNA-pCDNA3 to measure the binding between p21 and PCNA. HeLa cells were passaged in 3.5 cm dishes to 30% confluence, and after 16 hours, 500 ng each of the plasmid, mKG-N-p21-pCDNA3 and mKO-FM14-C-PCNA-pCDNA3, was transfected using the gene transfection reagent polyfect (QIAGEN) to co-express mKG-N-p21 and mKO-FM14-C-PCNA. Gene transfection was carried out according to the protocol of polyfect. After 24 hours of the gene transfection, green fluorescence images were obtained. The fluorescence image was obtained using the excitation filter BP460-480, the fluorescence filter BA495-540, and the dichroic mirror DM485. Excitation light was cut by 90% (10% transmission), and the exposure time was 1 second. An inverted microscope IX-71 (Olympus) with 40×Uapo/340 N.A. 1.35 lens (Olympus) was used. For image retrieval and analysis, MetaMolph (Nippon Roper Inc.) was used by binning 2 to obtain an image. Fluorescence image was obtained using a cooling CCD camera ORCA-ER (Hamamatsu Photonics Inc.). Expression images in the cytoplasm, nucleus and intranuclear luminance spots similar to those of the co-expression of mKO-FM14-N-p21-pCDNA3 and mKO-FM14-C-PCNA-pCDNA3 were obtained. However, since these images did not contain time information, the time and site relations of the complex of p21 and PCNA could not be determined from the images (FIG. 19)

In normal fluorescent imaging, the movement history of the fluorescently labeled compound is not recorded, and therefore images have to be obtained continuously throughout the passage of time. However, in the present invention, the history from the formation of protein complex to its movement can be obtained from the Ratio image, without the need for getting the images continuously.

The nucleotide sequences and amino acid sequences of the constructs used in the Example 9 and Comparative Example 1 described above are shown in the sequence listing as mentioned below.
Nucleotide sequence of mKO-FM14-N-p21 (SEQ ID NO: 57)
Amino acid sequence of mKO-FM14-N-p21 (SEQ ID NO: 58)
Nucleotide sequence of mKO-FM14-C-PCNA (SEQ ID NO: 59)
Amino acid sequence of mKO-FM14-C-PCNA (SEQ ID NO: 60)
Nucleotide sequence of mKG-N-p21 (SEQ ID NO: 61)
Amino acid sequence of mKG-N-p21 (SEQ ID NO: 62)

### ADVANTAGE OF THE INVENTION

Problems in GFP complementation system include that (1) it is not clear when the interaction (binding) has occurred because time information is not available; and that (2) in a case where the molecule moves after the binding, the history of the movement cannot be monitored because the history after the binding is not kept. These problems can be solved by the present invention. According to the present invention, a method and a kit for analyzing interaction between proteins are provided using a variant, in which fluorescence characteristic changes, for example, from green to orange, according to the passage of time and which can emit fluorescence by complementation while maintaining the fluorescence characteristic. For example, by using a measuring system for complementation by the variant which changes fluorescence characteristic from green to orange, it becomes possible to detect the passage of time by the ratio of green and orange. Further, for example, by observing the phenomenon that the interaction (binding) occurs on the cytoplasmic membrane and then the bound complex moves to the nucleus, through the ratio of green and orange, it becomes possible to monitor the history after the interaction (binding).

1. A method for analyzing interaction between a first test protein and a second test protein which comprises the steps of: splitting a fluorescent protein capable of emitting different color of fluorescence according to passage of time into an N-terminal fragment and a C-terminal fragment; allowing the first test protein to interact with the second test protein by making coexist a fusion protein of the N-terminal fragment with the first test protein and another fusion protein of the C-terminal fragment with the second test protein; and detecting the change in the fluorescent light due to the interaction.

2. The method of claim 1 wherein the fluorescent protein capable of emitting different color of fluorescence according to passage of time is a fluorescent protein having an amino acid sequence of SEQ ID NO: 2 in which the 70^{th} amino acid, proline, is substituted with another amino acid.

3. The method of claim 1 wherein the fluorescent protein capable of emitting different color of fluorescence according to passage of time is any of the following proteins:
(1) a fluorescent protein of an amino acid sequence represented by any of SEQ ID NO: 4, 6, 8, 10 and 12; or
(2) a fluorescent protein of an amino acid sequence represented by any of SEQ ID NO: 4, 6, 8, 10 and 12 wherein one or several amino acid are deleted, substituted and/or added, which has fluorescence characteristic which is changed from green color to orange color according to passage of time.

4. A kit for analyzing interaction between proteins, which includes a combination of a gene encoding an N-terminal fragment of a fluorescent protein having an amino acid sequence represented by SEQ ID NO: 2 in which the 70^{th} amino acid, proline, is substituted with another amino acid, and a gene encoding a C-terminal fragment of said fluorescent protein.

5. The kit of claim 4 wherein the fluorescent protein is any of the following proteins:
(1) a fluorescent protein of an amino acid sequence represented by any of SEQ ID NO: 4, 6, 8, 10 and 12; or
(2) a fluorescent protein of an amino acid sequence represented by any of SEQ ID NO: 4, 6, 8, 10 and 12 wherein one or several amino acid are deleted, substituted and/or added, which has fluorescence characteristic which is changed from green color to orange color according to passage of time.
